(19) United States
(12) Patent Application Publication
(10) Pub. No.: US 2024/0307025 A1
(43) Pub. Date: Sep. 19, 2024

(54) MACHINE LEARNING MODEL, PROGRAM, ULTRASOUND DIAGNOSTIC APPARATUS, IMAGE DIAGNOSTIC SYSTEM, IMAGE DIAGNOSTIC APPARATUS, AND TRAINING APPARATUS

(71) Applicant: Konica Minolta, Inc., Tokyo (JP)

(72) Inventors: Hiroaki MATSUMOTO, Kanagawa (JP); Yoshihiro TAKEDA, Tokyo (JP)

(21) Appl. No.: 18/602,510

(22) Filed: Mar. 12, 2024

(30) Foreign Application Priority Data
Mar. 14, 2023 (JP) 2023-039515

(51) Int. Cl. A61B 8/08; G06T 7/00; G06T 7/70; G06V 10/82; G16H 50/20

(52) U.S. Cl. CPC A61B 8/0883; A61B 8/5223; G06T 7/0012; G06T 7/70; G06V 10/82; G16H 50/20; G06T 2207/10132; G06T 2207/20081; G06T 2207/20084; G06T 2207/30048; G06V 2201/031; G06V 2201/07

(57) ABSTRACT

An image diagnostic technique using a machine learning model is disclosed. An aspect of the present disclosure relates to a machine learning model trained by using training data that includes first ultrasound image data based on a reception signal received by an ultrasound probe; first ground truth data that is first region information associated with a detection target of the first ultrasound image data; and second ground truth data that is first position information associated with the detection target of the first ultrasound image data or that is second region information based on the first position information.

### CROSS REFERENCE TO RELATED APPLICATIONS

The entire disclosure of Japanese Patent Application No. 2023-039515 filed on Mar. 14, 2023, is incorporated herein by reference in its entirety.

### BACKGROUND

### Technological Field

With the recent development of deep learning technology, machine learning models have come to be used for various purposes. For example, in the medical field, it has been proposed to use a machine learning model for image diagnosis of ultrasound image data or the like.

### SUMMARY

There are measurement items such as left ventricular ejection fraction (EF) and inferior vena cava (IVC) diameter as indicators for evaluating cardiac function, and accurate and highly reproducible measurements are desired. Currently, in manual EF measurement, the EF is calculated by a user tracing the endocardium in an ultrasound image. Further, in manual IVC diameter measurement, the IVC diameter is measured by the user designating the vascular wall of the inferior vena cava with reference to the hepatic vein. These manual measurements are complicated, and errors may occur due to user operations. In addition, captured images are different to each other, and an error may occur.

A semi-automatic EF measurement method is generally known. In this semiautomatic EF measurement method, the endocardium is automatically traced, but two points at the mitral annulus and one point at the cardiac apex needs to be specified by the user. Furthermore, in order for the user to designate two points on the mitral annulus and one point at the cardiac apex, it is necessary to freeze the ultrasound image and designate these points at the still image. As a result, it takes time and labor to perform the measurement, and it is difficult to perform the measurement in real time.

Further, unlike other organs, the heart is a part that moves greatly with beating. For example, in the calculation of the EF when the cardiac function is evaluated in real time, a sufficient left ventricular region cannot be recognized only by tracing the endocardium, and a technique for evaluating the cardiac function with high accuracy is required.

Inconsideration of the above-described problems, one object of the present disclosure is to provide an image diagnostic technology using a machine learning model.

To achieve at least one of the abovementioned objects, according to an aspect of the present invention, an aspect of the present disclosure relates to a machine learning model trained by using training data including first ultrasound image data based on a reception signal received by an ultrasound probe, first ground truth data that is first region information associated with a detection target of the first ultrasound image data, and second ground truth data that is first position information associated with the detection target of the first ultrasound image data or that is second region information based on the first position information.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a schematic diagram illustrating training processing and inference processing of a machine learning model according to an example of the present disclosure;

FIG. 2A is a diagram illustrating a left ventricular region in an exemplary ultrasound image, and FIG. 2B is a diagram illustrating an inferior vena cava in an exemplary ultrasound image;

FIG. 3 is a schematic diagram illustrating a machine learning model for a left ventricular region according to an example of the present disclosure;

FIG. 4 is a schematic diagram illustrating training processing and inference processing of a machine learning model according to another example of the present disclosure;

FIG. 5 is a schematic diagram illustrating training processing and inference processing of a machine learning model according to another example of the present disclosure;

FIG. 6 is a schematic diagram illustrating training processing and inference processing of a machine learning model according to another example of the present disclosure;

FIG. 7 is a schematic diagram illustrating an ultrasound diagnostic apparatus according to an example of the present disclosure;

FIG. 8 is a block diagram illustrating a hardware configuration of an ultrasound diagnostic apparatus according to an example of the present disclosure;

FIG. 9 is a block diagram illustrating a hardware configuration of a training apparatus and an image diagnostic apparatus according to an example of the present disclosure;

FIG. 10 is a block diagram illustrating a functional configuration of a training apparatus according to an example of the present disclosure;

FIG. 11A is a diagram illustrating training data of a left ventricular region according to an example of the present disclosure, and FIG. 11B is a diagram illustrating ground truth data of a region detection result according to an example of the present disclosure;

FIG. 12 is a schematic diagram illustrating a training processing of a machine learning model for EF measurement according to an example of the present disclosure;

FIG. 13 is a block diagram illustrating a functional configuration of an ultrasound diagnostic apparatus according to an example of the present disclosure;

FIG. 14 is a diagram illustrating a network architecture of a machine learning model for EF measurement according to an example of the present disclosure;

FIG. 15 is a schematic diagram illustrating target region detection for EF measurement according to an example of the present disclosure;

FIGS. 16A to C are schematic diagrams illustrating contour extraction processing according to an example of the present disclosure;

FIG. 17A is a diagram illustrating the training data of the inferior vena cava region according to an example of the present disclosure, and FIG. 17B is a diagram illustrating ground truth data of a region detection result according to an example of the present disclosure;

FIG. 18 is a schematic diagram illustrating training processing of a machine learning model for IVC diameter measurement according to an example of the present disclosure;

FIG. 19 is a diagram illustrating a network architecture of a machine learning model for IVC diameter measurement according to an example of the present disclosure;

FIG. 20A is a diagram illustrating training information of a left ventricular region according to an example of the present disclosure, and FIG. 20B is a diagram illustrating ground truth data of a region detection result according to an example of the present disclosure;

FIG. 21 is a schematic diagram illustrating a training processing of a machine learning model for EF measurement according to an example of the present disclosure;

FIG. 22 is a diagram illustrating a network architecture of a machine learning model for EF measurement according to an example of the present disclosure;

FIG. 23 is a schematic diagram illustrating target region detection for EF measurement according to an example of the present disclosure;

FIG. 24A is a diagram illustrating training data of an inferior vena cava region according to an example of the present disclosure, and FIG. 24B is a diagram illustrating ground truth data of a region detection result according to an example of the present disclosure;

FIG. 25 is a schematic diagram illustrating detection of a target region for IVC diameter measurement according to an example of the present disclosure; and

FIG. 26 is a diagram illustrating a network architecture of a machine learning model for IVC diameter measurement according to an example of the present disclosure.

### DETAILED DESCRIPTION OF EMBODIMENTS

Hereinafter, one or more embodiments of the present invention will be described with reference to the drawings. However, the scope of the invention is not limited to the disclosed embodiments.

### Outline of the Present Disclosure

The following examples disclose a training apparatus that trains a machine learning model for estimating a detection target region in an ultrasound image, and an ultrasound diagnostic apparatus, an image diagnostic apparatus, and an ultrasound diagnostic system that estimate a detection target region using the trained machine learning model and calculate indices (e.g., EF and IVC diameter) related to cardiac function.

More particularly, a machine learning model according to an example to be described later extracts region information indicating a part of a detection target region (for example, contours of a left ventricular lumen region, an inferior vena cava region, and the like) and position information indicating another part of the detection target region (for example, right and left annulus ends, a hepatic vein point, and the like) from an input ultrasound image. The ultrasound diagnostic apparatus, the image diagnostic apparatus, and the ultrasound diagnostic system estimate the detection target region based on the extracted region information and position information, and calculate an index for evaluating cardiac function based on the estimated detection target region. The machine learning model according to the present example can satisfactorily extract the detection target region from the beating heart as compared to directly extracting the detection target region.

### System Configuration

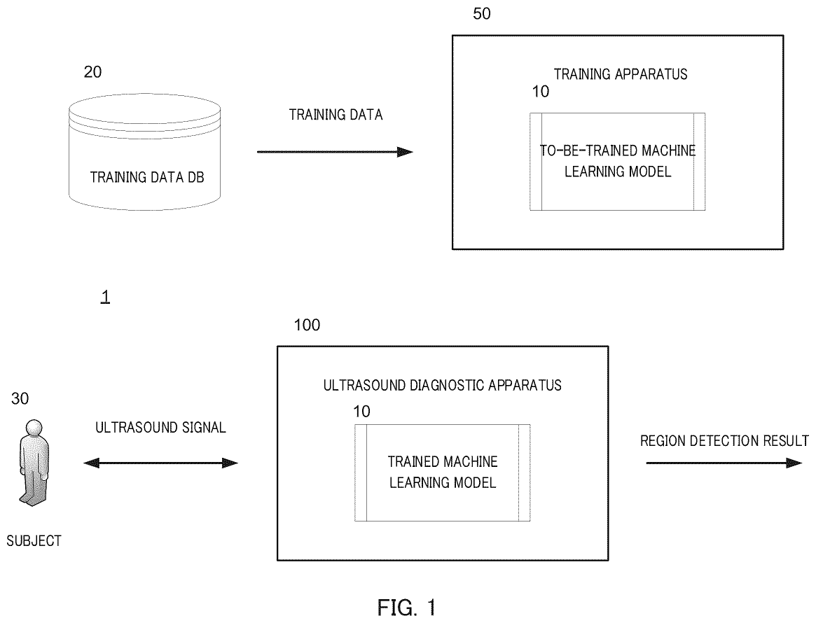

First, a system for implementing training and inference processing using a machine learning model according to an example of the present disclosure will be described. FIG. 1 is a schematic diagram illustrating processing and inference processing of a machine learning model according to an example of the present disclosure.

As illustrated in FIG. 1, the training apparatus 50 stores a to-be-trained machine learning model 10 to be trained, and trains the to-be-trained machine learning model 10 by using training data stored in a training data database (DB) 20. The to-be-trained machine learning model 10 may be implemented as any suitable type of machine learning model such as, for example, a neural network. For example, when the to-be-trained machine learning model 10 is implemented by a neural network, the training apparatus 50 may execute supervised learning using the training data acquired from the training data DB20 and update the parameters of the machine learning model 10 in accordance with any known training algorithm such as backpropagation.

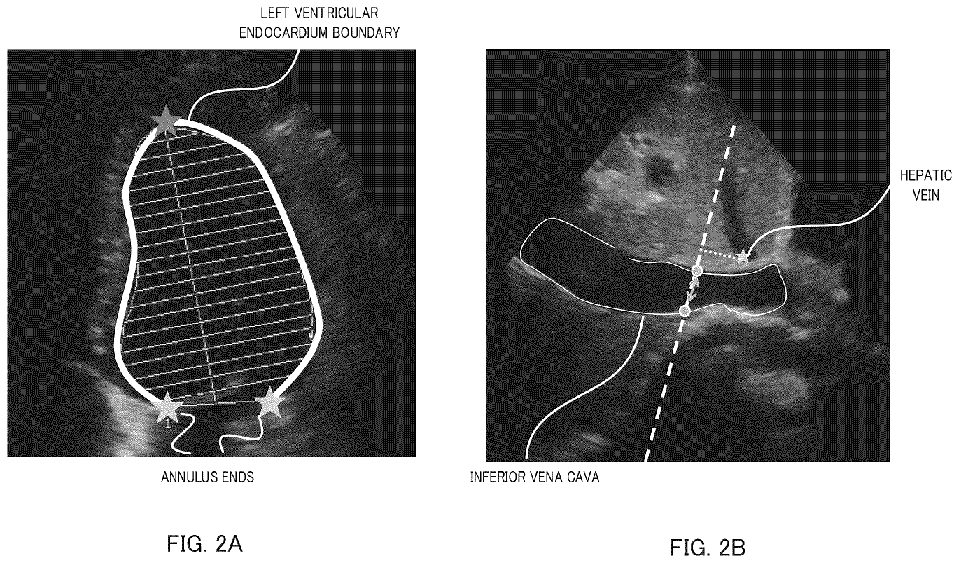

After the machine learning model 10 is trained, the trained machine learning model 10 may be stored in the ultrasound diagnostic apparatus 100, and the ultrasound diagnostic apparatus 100 may use the trained machine learning model 10 to estimate a detection result of a detection target region from ultrasound image data acquired by transmitting and receiving ultrasound signals to and from the subject 30 via the ultrasound probe. For example, when the machine learning model 10 has been trained to extract the left ventricular endocardium boundary and the right and left annulus ends from the ultrasound image of the heart, the ultrasound diagnostic apparatus 100 may extract the left ventricular endocardium boundary and the right and left annulus ends as illustrated FIG. 2A from the ultrasound image of the subject 30. The ultrasound diagnostic apparatus 100 estimates the left ventricular region in real time based on the region defined by the extracted left ventricular endocardium boundary and the left and right annulus ends, and estimates the left ventricular ejection fraction (EF) can be measured.

Alternatively, when the machine learning model 10 has been trained to extract the inferior vena cava and the hepatic veins from the ultrasound image of the heart, the ultrasound diagnostic apparatus 100 may extract the inferior vena cava region and the hepatic veins as illustrated in FIG. 2B from the ultrasound image of the subject 30. The ultrasound diagnostic apparatus 100 can measure, in real time, the diameter of the inferior vena cava (IVC) defined by the extracted inferior vena cava region and the hepatic vein.

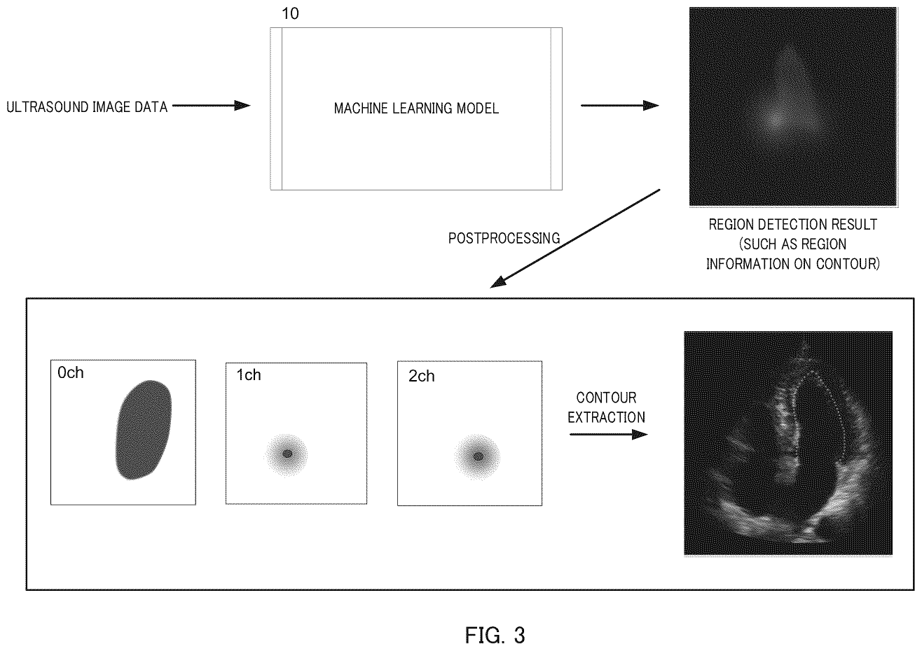

In one example, the machine learning model 10 for detecting the left ventricular region may extract region detection results of a plurality of channels from the ultrasound image data. In the example illustrated in FIG. 3, channel 0 indicates the boundary of the endocardium of the left ventricle, and channels 1 and 2 indicate the positions of the region detection results of the right and left annulus ends, respectively, in a weighted manner. The ultrasound diagnostic apparatus 100 may extract the contour or boundary of the left ventricular region based on the detection results of these three channels. Although not illustrated, the machine learning model 10 that detects the inferior vena cava and the hepatic vein can extract region detection results of a plurality of channels from the ultrasound image data in a similar manner. For example, a channel 0 may indicate an inferior vena cava region and a channel 1 may indicate a position of a hepatic vein, and the ultrasound diagnosis apparatus 100 may estimate an inferior vena cava (IVC) diameter based on detection results of the two channels.

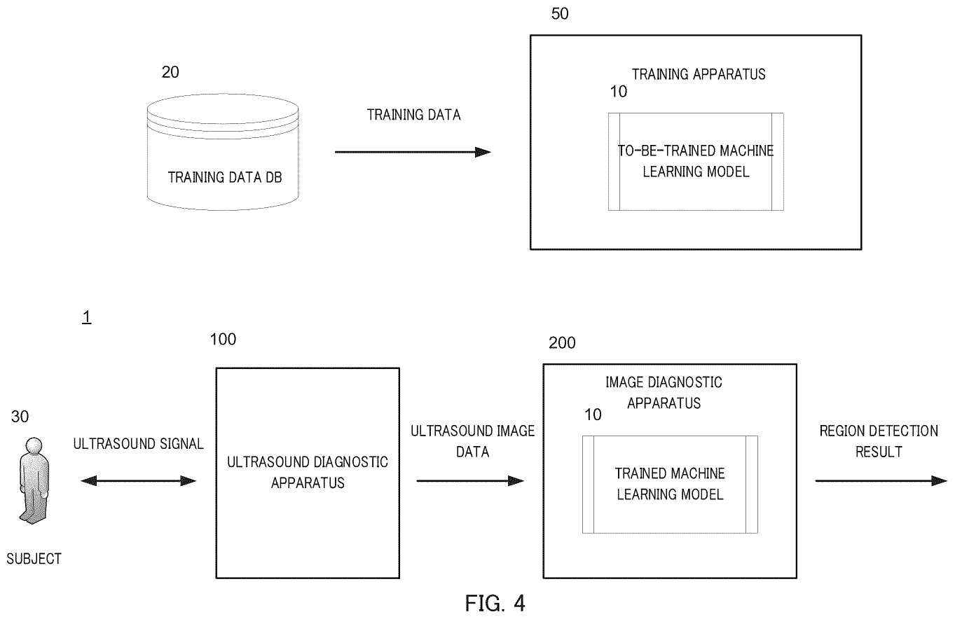

Although the system configuration according to an example of the present disclosure has been described with reference to FIG. 1, the system configuration according to the present disclosure is not limited thereto. FIG. 4 is a schematic diagram illustrating a training processing and an inference processing of machine learning model according to another example of the present disclosure. The illustrated example is the same as the example illustrated in FIG. 1 in that the training apparatus 50 trains the to-be-trained machine learning model 10, but in the present example, the ultrasound diagnostic system 1 may include an ultrasound diagnostic apparatus 100 and an image diagnostic apparatus 200, and the image diagnostic apparatus 200 may store the trained machine learning model 10 and estimate a region detection result from ultrasound image data acquired from the ultrasound diagnostic apparatus 100. According to the present example, even when the ultrasound diagnostic apparatus 100 does not include calculation resources for executing machine learning and the model 10, it is possible to execute machine learning model 10 by using more abundant calculation resources of the image diagnostic apparatus 200 implemented by a server or the like arranged on a network or the like.

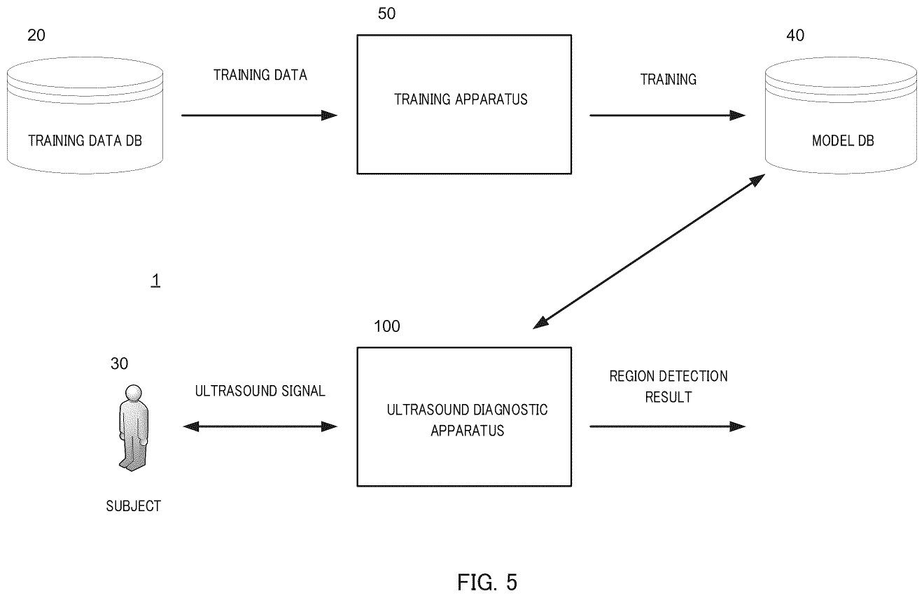

FIG. 5 is a schematic diagram illustrating a training processing and an inference processing of machine learning model according to another example of the present disclosure. In the illustrated example, the training apparatus 50 may train the to-be-trained machine learning model 10 stored in the model database (DB) 40 and store the trained machine learning model 10 in the model DB40. The ultrasound diagnostic apparatus 100 may access the model DB40 and utilize the trained machine learning model 10. For example, upon acquiring an ultrasound image data from the subject 30, the ultrasound diagnostic apparatus 100 may pass the acquired ultrasound image data to the trained machine learning model 10 stored in the model DB40, and acquire a region detection result from the model DB40. According to the present example, even when the ultrasound diagnostic apparatus 100 does not include a storage resource for storing the machine learning model 10, the machine learning model 10 stored in the model DB40 can be used.

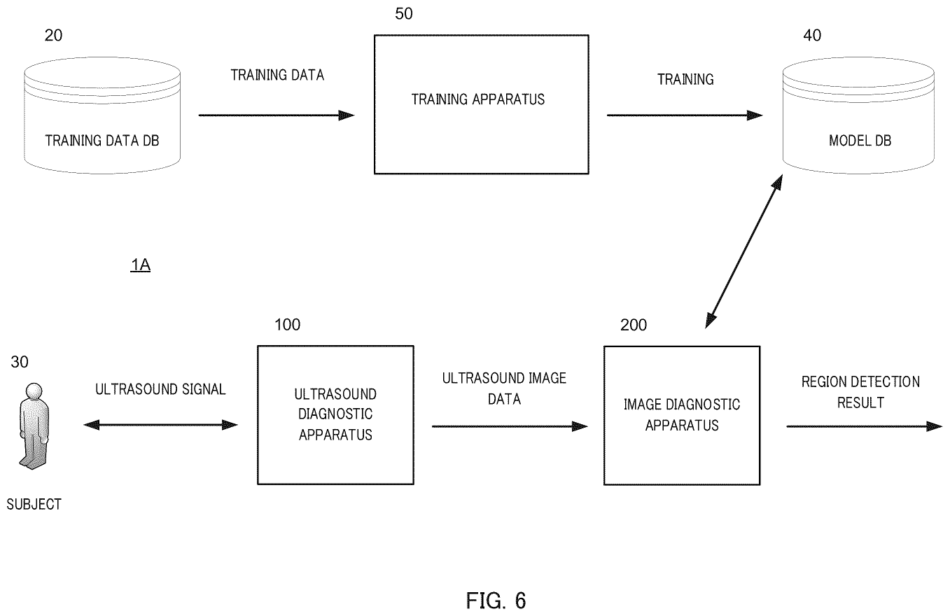

FIG. 6 is a schematic diagram illustrating a training processing and an inference processing of machine learning model according to another example of the present disclosure. In the illustrated example, the training apparatus 50 is similar to the example shown in FIG. 5 in that it trains the trained machine learning model 10 stored in the model DB40 and stores the trained machine learning model 10 in the model DB40, but in this example, the ultrasound diagnostic system 1A includes the ultrasound diagnostic apparatus 100 and the image diagnostic apparatus 200, and the image diagnostic apparatus 200 may pass the ultrasound image data acquired from the ultrasound diagnostic apparatus 100 to the trained machine learning model 10 of the model DB40 and acquire the region detection result from the model DB40. According to the present example, even when the ultrasound diagnostic apparatus 100 does not include calculation resources for executing the machine learning model 10 and/or does not include storage resources for storing the machine learning model 10, it is possible to use the machine learning model 10 by using more abundant calculation resources and storage resources of the model DB40 and the image diagnostic apparatus 200 implemented by a server or the like arranged on a network or the like.

### Hardware Configuration of Ultrasound Diagnostic Apparatus

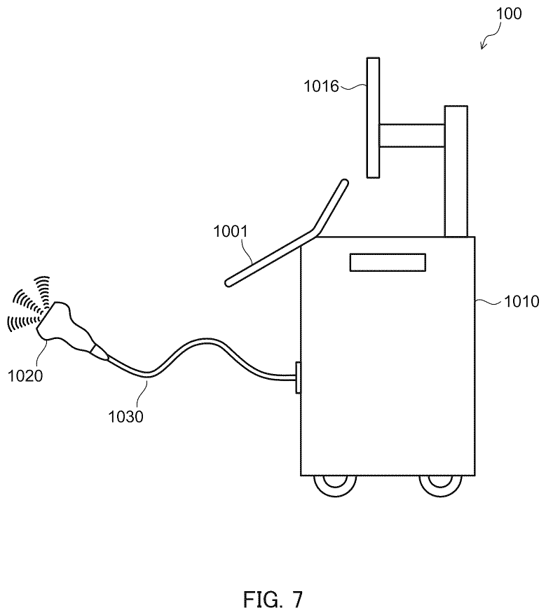

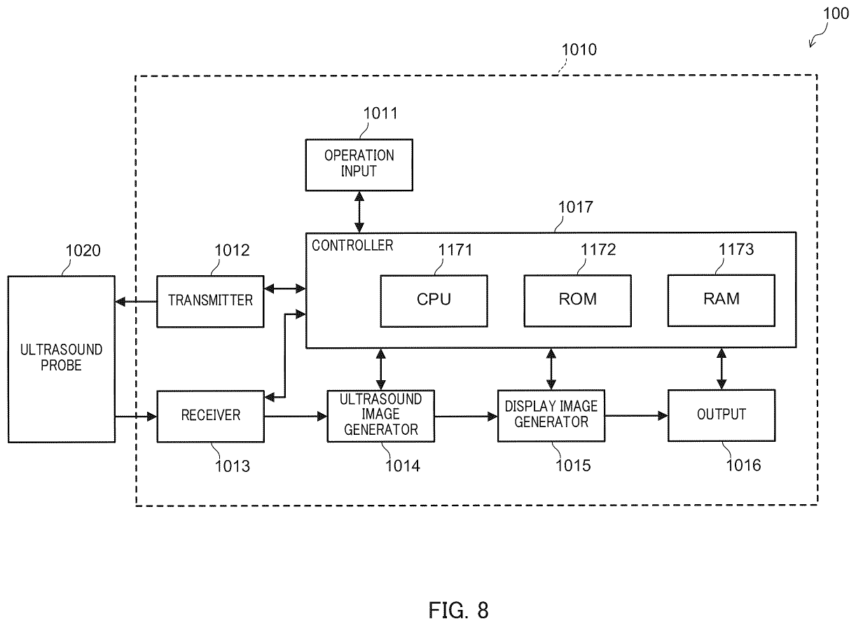

FIG. 7 is a diagram illustrating an example of an appearance of the ultrasound diagnostic apparatus 100. FIG. 8 is a block diagram illustrating an example of a configuration of a main part of a control system of the ultrasound diagnostic apparatus 100.

The ultrasound diagnostic apparatus 100 visualizes the shape or dynamics of the inside of the subject 30 as an ultrasound image. The ultrasound diagnostic apparatus 100 according to the present embodiment is used, for example, to capture an ultrasound image (i.e., a tomographic image) of a detection target site and perform an inspection on the detection target site.

As illustrated in FIG. 7, the ultrasound diagnostic apparatus 100 includes an ultrasound diagnostic apparatus body 1010 and an ultrasound probe 1020. The ultrasound diagnostic apparatus body 1010 and the ultrasound probe 1020 are connected to each other via a cable 1030.

The ultrasound probe 1020 functions as an acoustic sensor that transmits ultrasonic beams (for example, about 1 to 30 MHz) to the inside of the subject 30 (for example, a human body), receives ultrasonic echoes reflected in the subject 30 among the transmitted ultrasonic beams, and converts the ultrasonic echoes into electric signals.

The user brings the ultrasound beam transmission/reception surface of the ultrasound probe 1020 into contact with the body surface of the detection target region of the subject 30, operates the ultrasound diagnostic apparatus 100, and performs an inspection. As the ultrasound probe 1020, an arbitrary probe such as a convex probe, a linear probe, a sector probe, or a three dimensional probe can be applied.

The ultrasound probe 1020 is configured to include, for example, a plurality of transducers (e.g., piezoelectric elements) arranged in a matrix, and a channel switching device (e.g., a multiplexer) for controlling switching of on/off of a drive state of the plurality of transducers individually or in units of blocks (hereinafter referred to as “channels”).

Each transducer of the ultrasound probe 1020 converts a voltage pulse generated by the ultrasound diagnostic apparatus body 1010 (a transmitter 1012) into an ultrasonic beam, transmits the ultrasonic beam into the subject 30, receives an ultrasonic echo reflected inside the subject 30, converts the ultrasonic echo into an electric signal (hereinafter referred to as a “reception signal”), and outputs the electric signal to the ultrasound diagnostic apparatus body 1010 (a receiver 1013).

As illustrated in FIG. 8, the ultrasound diagnostic apparatus body 1010 includes an operation input 1011, the transmitter 1012, the receiver 1013, an ultrasound image generator 1014, a display image generator 1015, an output 1016, and a controller 1017.

The transmitter 1012, the receiver 1013, the ultrasound image generator 1014, and the display image generator 1015 are configured by dedicated or general-purpose hardware (electronic circuit) corresponding to each process, such as a digital signal processor (DSP), an application specific integrated circuit (ASIC), or a programmable logic device (PLD), and realize each function in cooperation with the controller 1017.

The operation input 1011 receives, for example, an input of a command instructing the start of diagnosis or the like or information on the subject 30. The operation input 1011 may include, for example, an operation panel including a plurality of input switches, a keyboard, a mouse, and the like. Note that the operation input 1011 may be formed by a touch panel provided integrally with the output 1016.

The transmitter 1012 is a transmitter that transmits a voltage pulse as a drive signal to the ultrasound probe 1020 according to an instruction of the controller 1017. The transmitter 1012 may include, for example, a high-frequency pulse oscillator, a pulse setter, and the like. The transmitter 1012 may adjust the voltage pulse generated by the high-frequency pulse oscillator to the voltage amplitude, the pulse width, and the transmission timing set by the pulse setter, and transmit the voltage pulse for each channel of the ultrasound probe 1020.

The transmitter 1012 includes a pulse setter for each of the plurality of channels of the ultrasound probe 1020, so that the voltage amplitude, pulse width, and transmission timing of a voltage pulse can be set for each of the plurality of channels. For example, the transmitter 1012 may change a target depth or generate different pulse waveforms by setting appropriate delay times for a plurality of channels.

The receiver 1013 is a receiver that performs reception processing on a reception signal related to an ultrasonic echo generated by the ultrasound probe 1020 in accordance with an instruction from the controller 1017. The receiver 1013 may include a preamplifier, an AD converter, and a reception beamformer.

The receiver 1013 amplifies a reception signal related to a weak ultrasonic echo for each channel by the preamplifier, and converts the reception signal into a digital signal by the AD converter. Then, the receiver 1013 can collect the reception signals of the plurality of channels into one by performing phasing addition on the reception signals of the respective channels in the reception beamformer to obtain acoustic line data.

The ultrasound image generator 1014 acquires the reception signals (acoustic line data) from the receiver 1013 and generates an ultrasound image (i.e., a tomographic image) of the inside of the subject 30.

For example, when the ultrasound probe 1020 transmits a pulsed ultrasonic beam toward the depth direction, the ultrasound image generator 1014 accumulates, in the line memory, the signal intensity of the ultrasonic echoes detected thereafter in a temporally continuous manner. Then, as the ultrasonic beam from the ultrasound probe 1020 scans the inside of the subject 30, the ultrasound image generator 1014 sequentially accumulates the signal intensity of the ultrasonic echo at each scanning position in the line memory, and generates two dimensional data in units of frames. The ultrasound image generator 1014 may then convert the signal intensity of the two dimensional data into a luminance value, to generate an ultrasound image representing a two dimensional structure in a cross section including the transmission direction of the ultrasound and the scanning direction of the ultrasonic wave.

Note that the ultrasound image generator 1014 may include, for example, an envelope detection circuit that performs envelope detection on the reception signal acquired from the receiver 1013, a logarithmic compression circuit that performs logarithmic compression on the signal intensity of the reception signal detected by the envelope detection circuit, and a dynamic filter that removes a noise component included in the reception signal by a band-pass filter whose frequency characteristics are changed according to the depth.

The display image generator 1015 acquires the data of the ultrasound image from the ultrasound image generator 1014 and generates a display image including a display region of the ultrasound image. Then, the display image generator 1015 transmits the data of the generated display image to the output 1016. The display image generator 1015 may sequentially update the display image each time a new ultrasound image is acquired from the ultrasound image generator 1014, and cause the output 1016 to display the display image in a moving image format.

Furthermore, the display image generator 1015 may generate, in accordance with an instruction from the controller 1017, a display image (with an image in which time-series data to be detected is graphically displayed is embedded therein) in a display region together with an ultrasound image.

Note that the display image generator 1015 may generate the display image after performing predetermined image processing, such as coordinate conversion processing and data interpolation processing, on the ultrasound image output from the ultrasound image generator 1014.

In accordance with an instruction from the controller 1017, the output 1016 acquires data of a display image from the display image generator 1015 and outputs the display image. For example, the output 1016 may be configured by a liquid crystal display, an organic EL display, a CRT display, or the like, and may display a display image.

The controller 1017 performs overall control of the ultrasound diagnostic apparatus 100 by controlling each of the operation input 1011, the transmitter 1012, the receiver 1013, the image generator 1014, the display image generator 1015, and the output 1016 in accordance with their functions.

The controller 1017 may include a central processing unit (CPU) 1171 as an arithmetic/control device, a read only memory (ROM) 1172 and a random access memory (RAM) 1173 as main storage devices, and the like. The ROM1172 stores basic programs and basic setting information. The CPU1171 reads a program corresponding to processing content from the ROM172, stores the program in the RAM1173, and executes the stored program, thereby centrally controlling the operation of each functional block (the transmitter 1012, the receiver 1013, the ultrasound image generator 1014, the display image generator 1015, and the output 1016) of the ultrasound diagnostic apparatus body 1010.

### Hardware Configuration of Training Apparatus and Image Processing Apparatus

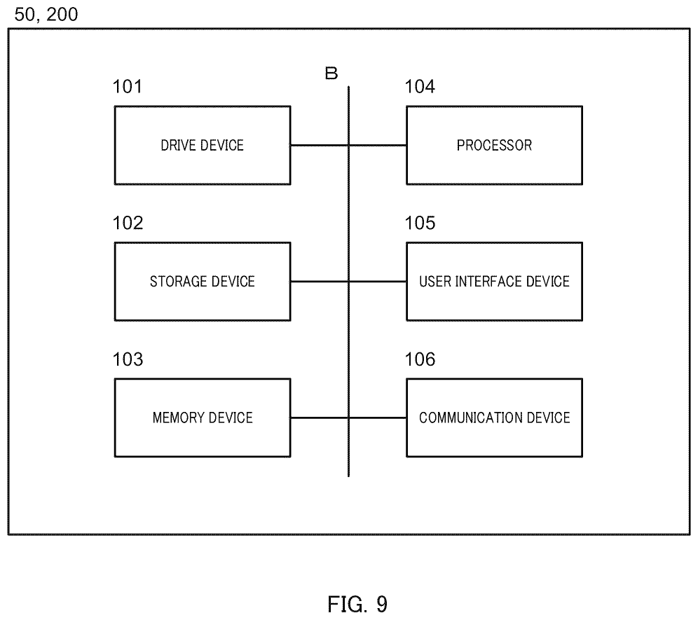

Next, a hardware configuration of the training apparatus 50 and the image processing apparatus 200 according to an example of the present disclosure will be described with reference to FIG. 9. FIG. 9 is a block diagram illustrating a hardware configuration of the training apparatus 50 and the image processing apparatus 200 according to an example of the present disclosure.

The training apparatus 50 and the image processing apparatuses 200 may each be implemented by a computing apparatus such as a server, a personal computer, a smartphone, or a tablet, and may have, for example, a hardware configuration as illustrated in FIG. 9. That is, the training apparatus 50 and the image processing apparatus 200 each include a drive device 101, a storage device 102, a memory device 103, a processor 104, a user interface (UI) device 105, and a communication device 106, which are interconnected via a bus B.

The programs or instructions for implementing various functions and processes, which will be described later, in the training apparatus 50 and the image processing apparatus 200 may be stored in removable storage media, such as a compact disk-read only memory (CD-ROM) and a flash memory. When the storage medium is set in the drive device 101, a program or an instruction is installed in the storage device 102 or the memory device 103 from the storage medium via the drive device 101. Note, however, that the program or the instructions are not necessarily installed from the storage media but may be downloaded from any external apparatus via a network or the like.

The storage device 102 is implemented by a hard disk drive or the like, and stores, together with an installed program or instruction, a file, data, or the like used for execution of the program or instruction.

The memory device 103 is implemented by a random access memory, a static memory, or the like, and when a program or an instruction is activated, reads the program, the instruction, data, or the like from the storage device 102 and stores the read program, instruction, data, or the like. The storage device 102, the memory device 103, and the removable storage medium may be collectively referred to as a non-transitory storage medium.

The processor 104 may be implemented by at least one of central processing unit (CPU), graphics processing unit (GPU), processing circuitry, and the like, which may be comprised of one or more processor cores, and executes various functions and processing of the training apparatus 50 and the image processing apparatuses 200, which will be described later, in accordance with programs and instructions stored in the memory device 103, data such as parameters necessary to execute the programs or instructions, and/or the like.

The user interface (UI) device 105 may include input devices such as a keyboard, a mouse, a camera, and a microphone, output devices such as a display, a speaker, a headset, and a printer, and input/output devices such as a touch panel, and implements an interface between the user and the training apparatus 50 and the image processing apparatus 200. For example, the user operates a graphical user interface (GUI) displayed on the display or the touch panel with a keyboard, a mouse, or the like to operate the training apparatus 50 and the image processing apparatus 200.

The communication device 106 is implemented by various communication circuits that execute wired and/or wireless communication processing with an external device or a communication network such as the Internet, a local area network (LAN), or a cellular network.

However, the above-described hardware configuration is merely an example, and the training apparatus 50 and the image processing apparatuses 200 according to the present disclosure may be implemented by any other appropriate hardware configuration.

### First Example

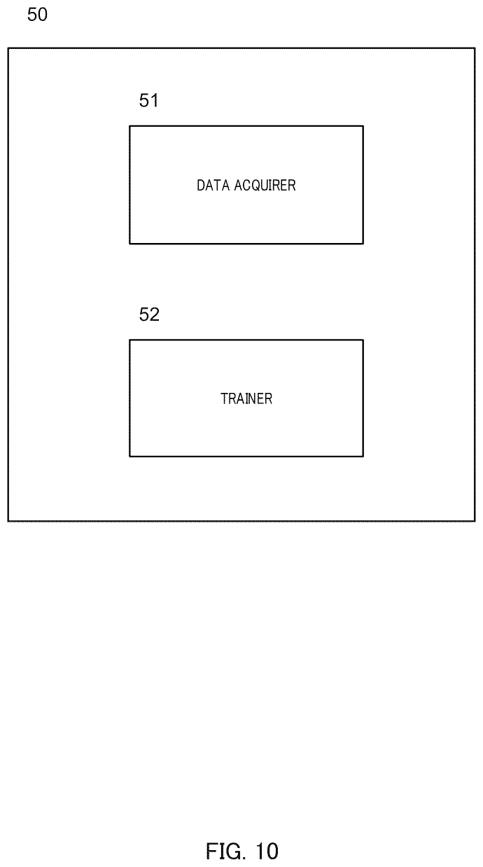

Next, a training apparatus 50 according to an example of the present disclosure will be described. The present example will be described focusing on the to-be-trained machine learning model 10 for detecting the left ventricular region to be used for EF measurement. FIG. 10 is a block diagram illustrating a functional configuration of the training apparatus 50 according to an example of the present disclosure. As illustrated in FIG. 10, the training apparatus 50 includes a data acquirer 51 and a trainer 52.

The data acquirer 51 acquires training data for a to-be-trained machine learning model 10. Specifically, the data acquirer 51 acquires training data including ultrasound image data and ground truth data including region information associated with a detection target of the ultrasound image data and/or position information associated with the detection target of the ultrasound image data or region information based on the position information.

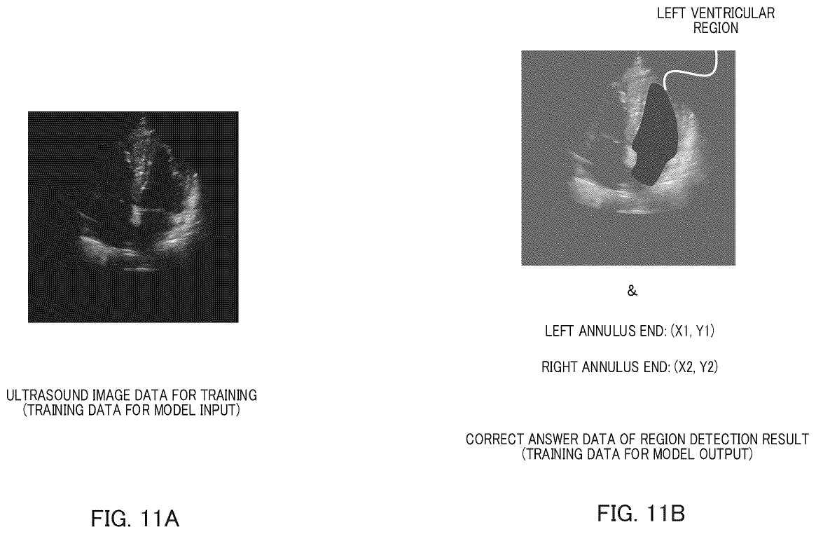

For example, the data acquirer 51 may acquire, from the training data DB20, ultrasound image data that represents the heart, as illustrated in FIG. 11A, as training data for input to the machine learning model 10. Furthermore, as the output-use training data to be output from the machine learning model 10, the data acquirer 51 may acquire, from the training data DB20, the data indicating the left ventricular region in the ultrasound image data as illustrated in FIG. 11B, and the coordinates indicating the positions of the left annulus end and the right annulus end. The data indicating the left ventricular region is a data set associated with information on the certainty factor (e.g., a numerical value indicating the certainty factor that it is the left ventricular region corresponding to the pixel block at the center of the ultrasound image data). Note that a pixel block is each divided region when an image is divided into a plurality of regions, and may be constituted by a pixel group including a plurality of pixels, or may be constituted by one pixel. Note that the data acquirer 51 may preprocessing the acquired ultrasound image data for training, as necessary. The preprocessing may include, for example, contrast change, noise removal, and the like.

Furthermore, the data acquirer 51 may expand the training data acquired from the training data DB20 to increase the training data. For example, the data acquirer 51 may perform enlargement/reduction, position change, deformation, and the like on the training ultrasound image acquired from the training data DB20.

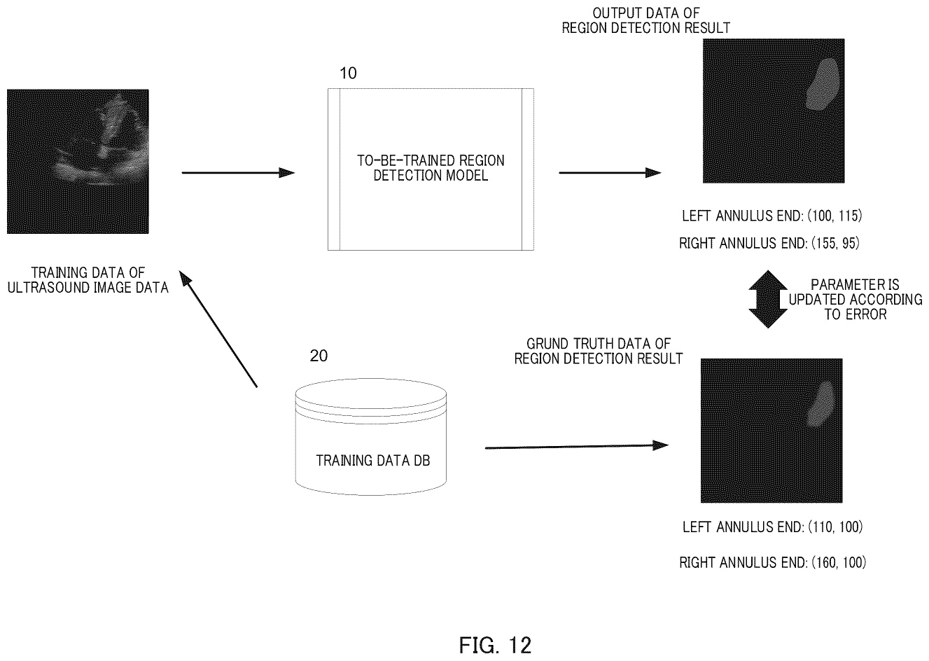

The trainer 52 compares the output result of the data indicating the left ventricular region output from the machine learning model 10 of the training target and the coordinates indicating the positions of the left annulus end and the right annulus end with the ground truth data, and updates the parameters of the machine learning model 10 in accordance with the error between the output result and the ground truth data. As illustrated in FIG. 12, the trainer 52 may input the ultrasound image data for training into the machine learning model 10 and acquire, from the machine learning model 10, the data indicating the left ventricular region in the ultrasound image data and the coordinates indicating the positions of the left annulus end and the right annulus end. For example, in the example illustrated in FIG. 12, the machine learning model 10 to be trained outputs data indicating the left ventricular region as illustrated and coordinates (100, 115) and (155, 95) indicating the positions of the left annulus end and the right annulus end. The trainer 52 compares the detection result with the ground truth data of the data indicating the left ventricular region and the coordinates (110, 100) and (160, 100) indicating the positions of the left annulus end and the right annulus end in the input ultrasound image data for training, and updates the parameters of the machine learning model 10 according to the error between the detection result and the ground truth data. For example, in a case where the machine learning model 10 is implemented by a convolutional neural network, the trainer 52 may continue to adjust the parameters of the machine learning model 10 according to the error between the output result and the ground truth data in accordance with the back propagation method until a predetermined termination condition is satisfied.

After the training of the machine learning model 10 is completed in this way, the trained machine learning model 10 may be provided to the ultrasound diagnostic apparatus 100. Alternatively, the trained machine learning model 10 may be provided to a model DB40 and/or the image diagnostic apparatus 200.

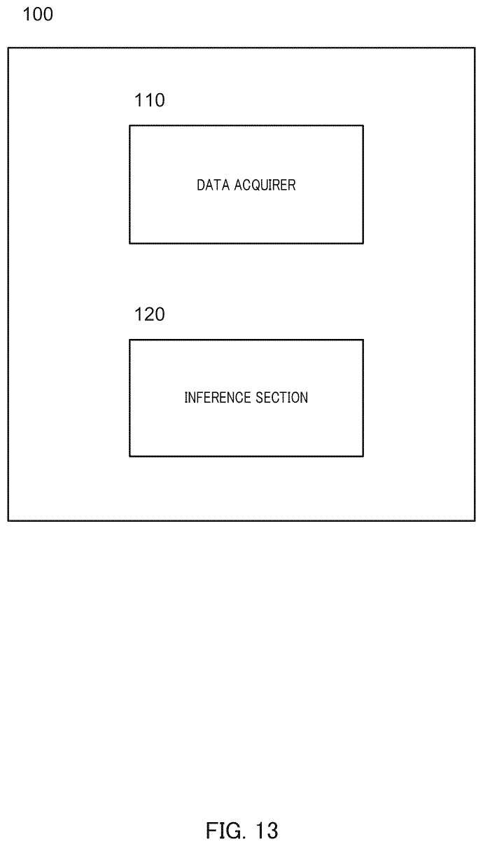

Next, an ultrasound diagnostic apparatus 100 according to an example of the present disclosure will be described. The ultrasound diagnostic apparatus 100 uses the machine learning model 10 trained by the training apparatus 50 to perform ultrasound diagnosis based on ultrasound signals transmitted to and received from the subject 30. FIG. 13 is a block diagram illustrating a functional configuration of the ultrasound diagnostic apparatus 100 according to an example of the present disclosure. As shown in FIG. 13, the ultrasound diagnostic apparatus 100 includes a data acquirer 110 and an inference section 120.

The data acquirer 110 acquires ultrasound image data of an inference target. Specifically, the data acquirer 110 acquires the ultrasound image data generated based on the reception signal received from the subject 30 by the ultrasound probe 1120. Note that as necessary, the data acquirer 110 may perform preprocessing, such as noise suppression, contrast normalization, and image resizing, on the acquired ultrasound image data for input to the trained machine learning model 10.

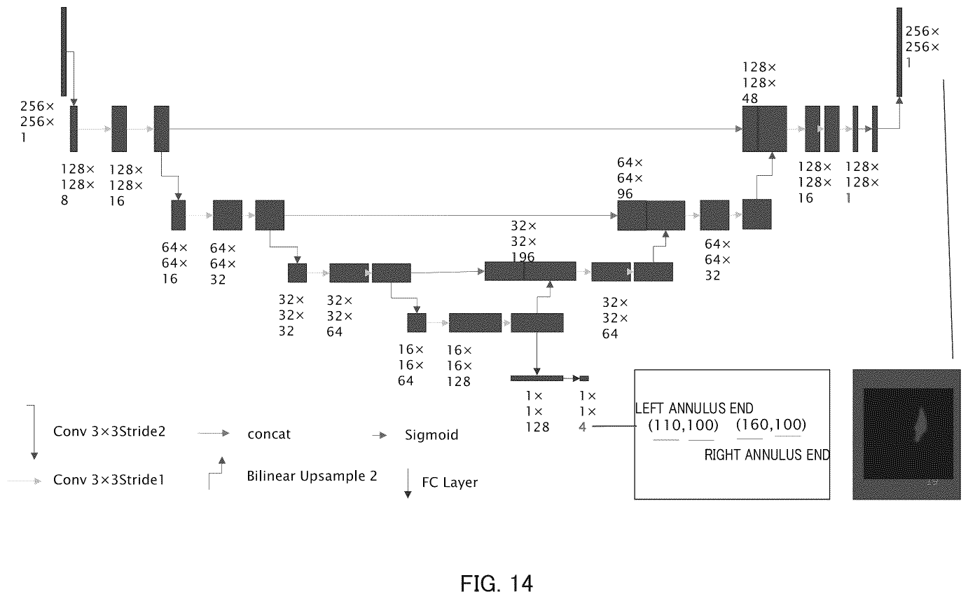

The inference section 120 inputs the ultrasound image data of an inference target to the trained machine learning model 10 and acquires an inference result. Specifically, the inference section 120 inputs the ultrasound image data of an inference target into the trained machine learning model 10, and acquires, from the machine learning model 10, data indicating the contour of the left ventricular region and the coordinates indicating the positions of the left annulus end and the right annulus end. For example, in a case where the trained machine learning model 10 is implemented as a U-net type convolutional neural network as illustrated in FIG. 14, the inference section 120 inputs ultrasound image data of an inference target to the input layer of the convolutional neural network and acquires data indicating the left ventricular region and coordinates indicating the positions of the left and right annulus ends from the output layer. In the illustrated example, data indicating the left ventricular region, the coordinates (110, 100) of the left annulus end, and the coordinates (160, 100) of the right annulus end are output.

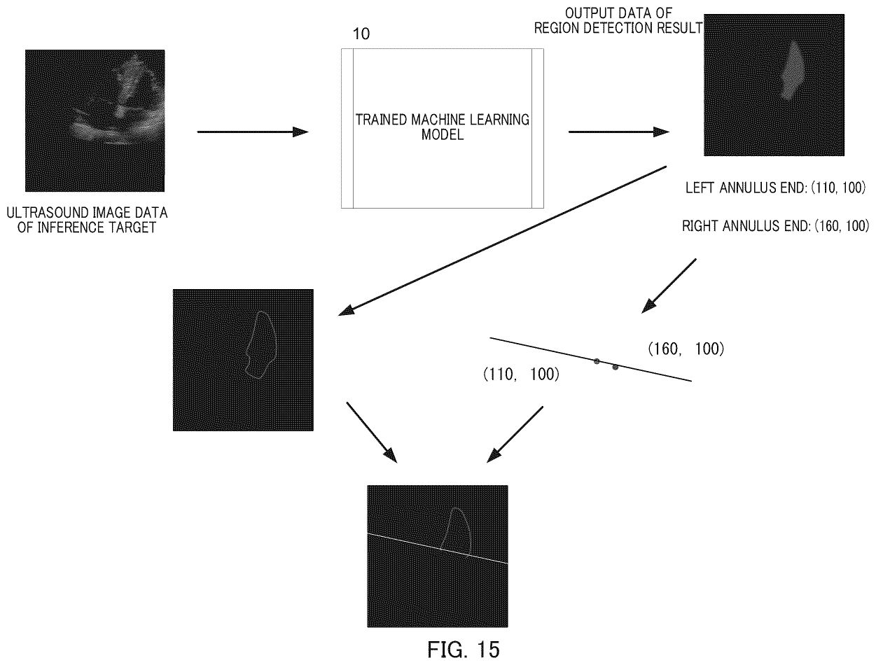

The inference section 120 determines a measurement target region based on data indicating the left ventricular region estimated by the machine learning model 10, the coordinates of the left annulus end, and the coordinates of the right annulus end. Specifically, when the data indicating the left ventricular region, the coordinates (110, 100) of the left annulus end, and the coordinates (160, 100) of the right annulus end are acquired from the machine learning model 10, as illustrated in FIG. 15, the inference section 120 derives a straight line connecting the left annulus end and the right annulus end, and superimposes the straight line on the delineated left ventricular region. Then, the inference section 120 can determine a region surrounded by the rendered left ventricular region and the straight line as a measurement target region, and estimate the left ventricle ejection fraction based on the volume of the measurement target region.

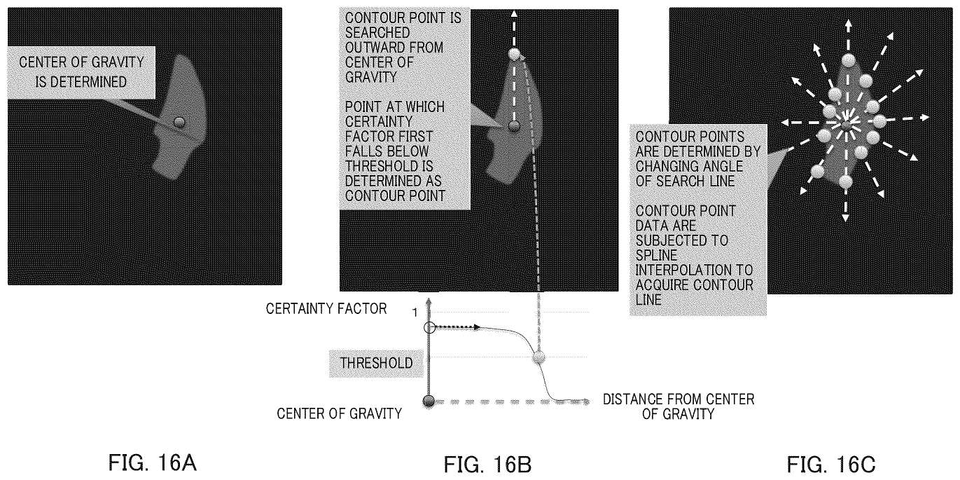

Here, the data indicating the contour line of the left ventricular region may be acquired, for example, according to a procedure as illustrated in FIGS. 16A to C. That is, as illustrated in FIG. 16A, the center of gravity of the left ventricular region is first determined, and then contour points are searched for outward from the determined center of gravity. Next, as illustrated in FIG. 16B, a point at which the certainty factor as an output result first falls below a threshold value may be determined as a contour point. As illustrated in FIG. 16C, the processing variously changes the angle of the search line extending from the center of gravity, and thus the contour points on respective search lines are determined. Then, these contour point data are subjected to spline interpolation to acquire a contour line. Further, the volume of the measurement target region may be estimated based on the contour line determined from the data indicating the left ventricular region in this manner. For example, the capacity of the measurement target region may be derived according to the Modified Simpson method (disk method). Specifically, the major axis (L) of two cross sections of the apical 2-chamber or 4-chamber image is equally divided into 20 disks, minor axis inner diameters (a₁ and b₁) orthogonal to the major axis are obtained, and the volume is calculated from the sum of the cross-sectional areas of the disks. Assuming that each disk has an elliptical shape, the left ventricular cavity area (V) is determined. That is, the left ventricular cavity area (V) to be measured can be calculated by the following Equation.

$$V = \frac{π}{4} \times ( {a}_{1} {b}_{1} + {a}_{2} {b}_{2} + … + {a}_{i} {b}_{i} ) \times \frac{L}{20}$$

Note that, in another example, the machine learning model 10 that directly estimates the measurement target region itself may be generated. However, estimating the measurement target region itself by the machine learning model 10 may generally degrade the estimation accuracy.

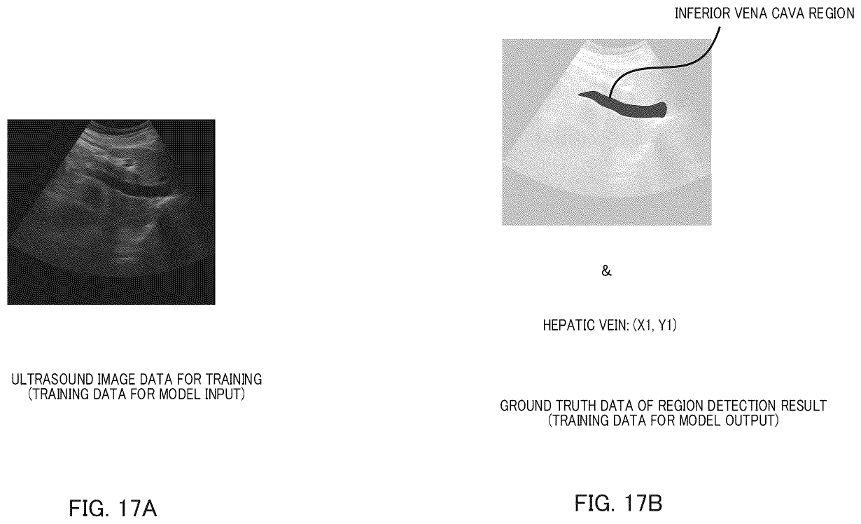

The machine learning model 10 that estimates the coordinates related to the detection target region and the specific position described above is not limited to EF measurement, and may be used for IVC diameter measurement. First, with respect to the training processing, for example, the data acquirer 51 may acquire, from the training data DB20, ultrasound image data representing the inferior vena cava, as illustrated in FIG. 17A, as training data for input to the machine learning model 10. Furthermore, as illustrated in FIG. 17B, the data acquirer 51 may acquire, from the training data DB20, data that indicates the region of the inferior vena cava region in the ultrasound image and coordinates that indicate the positions of the hepatic veins, as training data-for-output to be output from the machine learning model 10. Note that the data acquirer 51 may perform preprocessing and/or data expansion on the acquired ultrasound image data for training, as necessary.

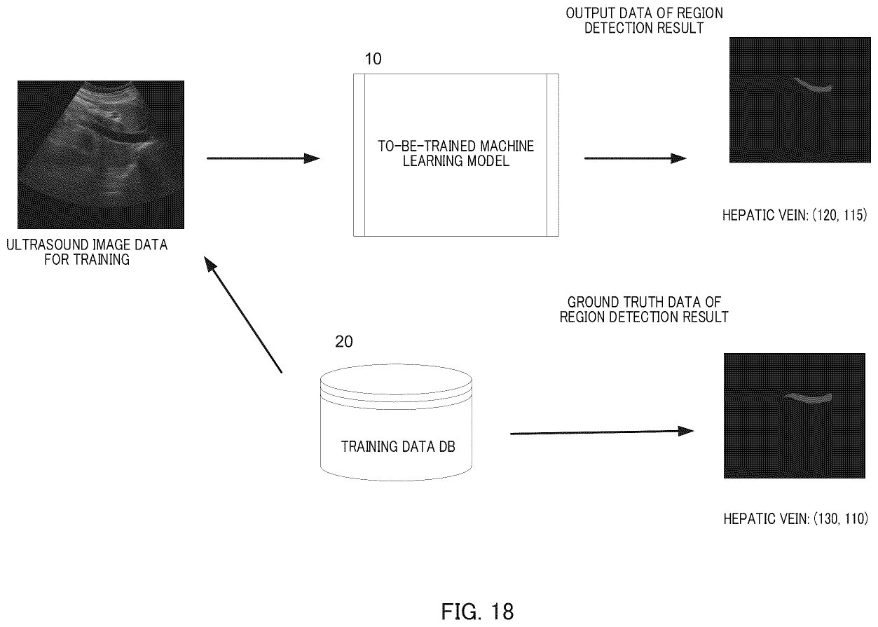

The trainer 52 may input the ultrasound image data for training into the machine learning model 10 and acquire, from the machine learning model 10, the data indicating the region of the inferior vena cava region in the ultrasound image data and the coordinates indicating the position of the hepatic vein. For example, as illustrated in FIG. 18, the ultrasound image data for training stored in the training data DB20 may be input to the machine learning model 10 to be trained, and the data indicating inferior vena cava region and the coordinates (120, 115) indicating the position of the hepatic vein may be output as the detection result. The trainer 52 compares the detection result with the ground truth data of the coordinates (130, 110) indicating the positions of the inferior vena cava region and the hepatic vein in the input ultrasound image data for training, and updates the parameters of the machine learning model 10 according to the error between the detection result and the ground truth data.

For example, in a case where the machine learning model 10 is implemented by a convolutional neural network, the trainer 52 may continue to adjust the parameters of the machine learning model 10 in accordance with the error between the output result and the ground truth data in accordance with the back propagation method until a predetermined termination condition is satisfied. After the training of the machine learning model 10 is completed in this way, the trained machine learning model 10 may be provided to the ultrasound diagnostic apparatus 100. Alternatively, the trained machine learning model 10 may be provided to a model DB40 and/or the image diagnostic apparatus 200.

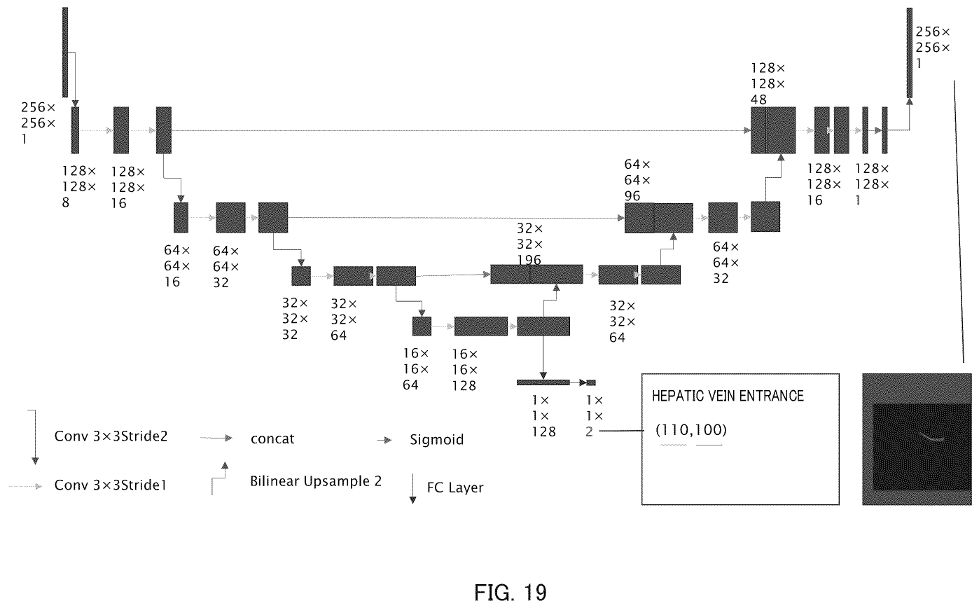

Next, in the inference processing, the data acquirer 110 acquires ultrasound image data generated based on a reception signal received from the subject 30 by the ultrasound probe 1120. As necessary, the data acquirer 110 may perform preprocessing on the acquired ultrasound image data for input to the trained machine learning model 10. The inference section 120 inputs the ultrasound image data of an inference target to the trained machine learning model 10, and acquires data indicating the inferior vena cava region and coordinates indicating the position of the hepatic vein from the machine learning model 10. For example, when the trained machine learning model 10 is implemented as a U-net type convolutional neural network as illustrated in FIG. 19, the inference section 120 inputs the ultrasound image data of an inference target to the input layer of the convolutional neural network, and acquires data indicating the inferior vena cava region and coordinates indicating the position of the hepatic vein from the output layer. In the illustrated example, data indicating the inferior vena cava region and the coordinates (110, 100) of the hepatic vein are output. Then, the inference section 120 can estimate the IVC diameter from the region determined based on the data of the inferior vena cava region estimated by the machine learning model 10 and the coordinates indicating the position of the hepatic vein. Specifically, the inference section 120 sets a measurement point search line based on the distance from the preset hepatic vein position, obtains two intersection points of the measurement point search line and the estimated inferior vena cava region, and estimates the distance between the intersection points as the IVC diameter. The inference section 120 may obtain each of distances between intersection points corresponding to the plurality of measurement point search lines at different angles, and may determine, as the distance of the IVC diameter, the distance between the intersection points at which the distance is minimum. Furthermore, the inference section 120 may determine the angle of the measurement point search line based on the data on the inferior vena cava region, and estimate the IVC diameter from the determined measurement line search line.

### Second Example

Next, a target region detection processing using the machine learning model 10 according to the second example of the present disclosure will be described. Upon receiving the ultrasound image data, the machine learning model 10 according to the first example detects the data indicating the left ventricular region or the inferior vena cava and the coordinates indicating the positions of the right and left annulus ends or the hepatic vein in the ultrasound image data. On the other hand, when receiving ultrasound image data, the machine learning model 10 according to the second example detects data indicating the left ventricular region or the inferior vena cava in the ultrasound image data and data indicating regions where the right and left annulus ends or the hepatic veins are present. For example, the data indicating such a region may be data in the form of a heat map representing the certainty factor of the position of the detection target. The heat map data may be data in any form indicating a certainty factor or a probability that the detection target exists at each position on the map.

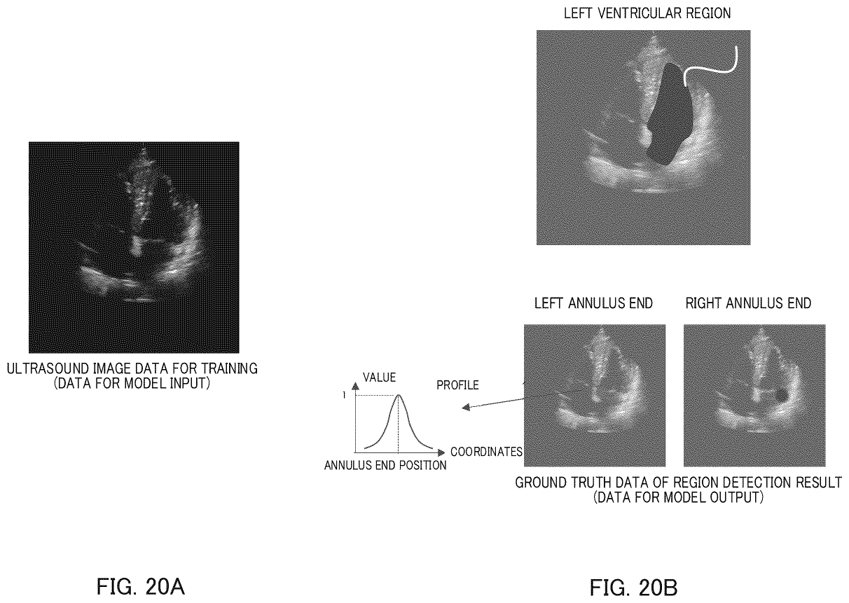

For the training processing, the data acquirer 51 may acquire, from the training data DB20, ultrasound image data representing the heart as illustrated in FIG. 20A, as training data for input to the machine learning model 10. Furthermore, the data acquirer 51 may acquire, from the training data DB20, as training data-for-output to be output from the machine learning model 10, data that indicates a left ventricular region in ultrasound image data as illustrated in FIG. 20B, and heat map data that indicates the certainty factor of the positions of the left and right annulus ends. As necessary, the data acquirer 51 may preprocessing the acquired ultrasound image data for training. The preprocessing may include, for example, contrast modification, noise removal, etc.

Furthermore, the data acquirer 51 may expand the training data acquired from the training data DB20 to increase the training data. For example, the data acquirer 51 may perform enlargement/reduction, position change, deformation, and the like on the training ultrasound image acquired from the training data DB20. In addition, the data acquirer 51 may correct the data in the heat map format indicating the certainty factor of the position of the detection target after the data expansion within a range in which the peak position of the certainty factor does not change. Specifically, in the case of the heat map according to the distance from the peak position of the certainty factor, the data acquirer 51 corrects the heat map changed by the data expansion to the heat map according to the distance before the data expansion and the reference. Accordingly, the value of the certainty factor of the heat map can reflect the distance from the position of the detection target.

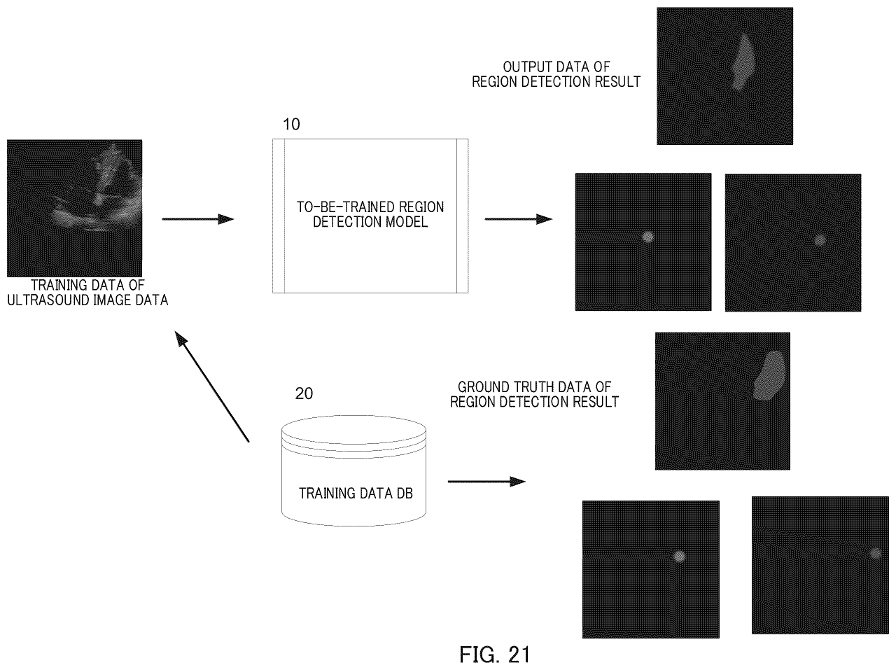

The trainer 52 trains the machine learning model 10 to be trained by using the training data. To be more specific, the trainer 52 may input the ultrasound image for training to the machine learning model 10, and may acquire, from the machine learning model 10, the left ventricular region in the ultrasound image, and the heat map indicating the degrees of certainty of the positions of the left annulus end and the right annulus end. As shown in FIG. 21, the trainer 52 compares the left ventricular region data output from the machine learning model 10 to be trained and the data indicating the certainty factors of the positions of the left annulus end and the right annulus end with the ground truth data, and updates the parameters of the machine learning model 10 in accordance with the error between the output result and the ground truth data. For example, in a case where the machine learning model 10 is implemented by a convolutional neural network, the trainer 52 may continue to adjust the parameters of the machine learning model 10 according to the error between the output result and the ground truth data in accordance with the back propagation method until a predetermined termination condition is satisfied.

After the training of the machine learning model 10 is completed in this way, the trained machine learning model 10 may be provided to the ultrasound diagnostic apparatus 100. Alternatively, the trained machine learning model 10 may be provided to a model DB40 and/or the image diagnostic apparatus 200.

In the inference processing, the data acquirer 110 acquires ultrasound image data generated based on a reception signal received from the subject 30 by the ultrasound probe 1120. The data acquirer 110 may perform necessary preprocessing, such as noise suppression, contrast normalization, and image resizing, on the acquired ultrasound image data for input to the trained machine learning model 10.

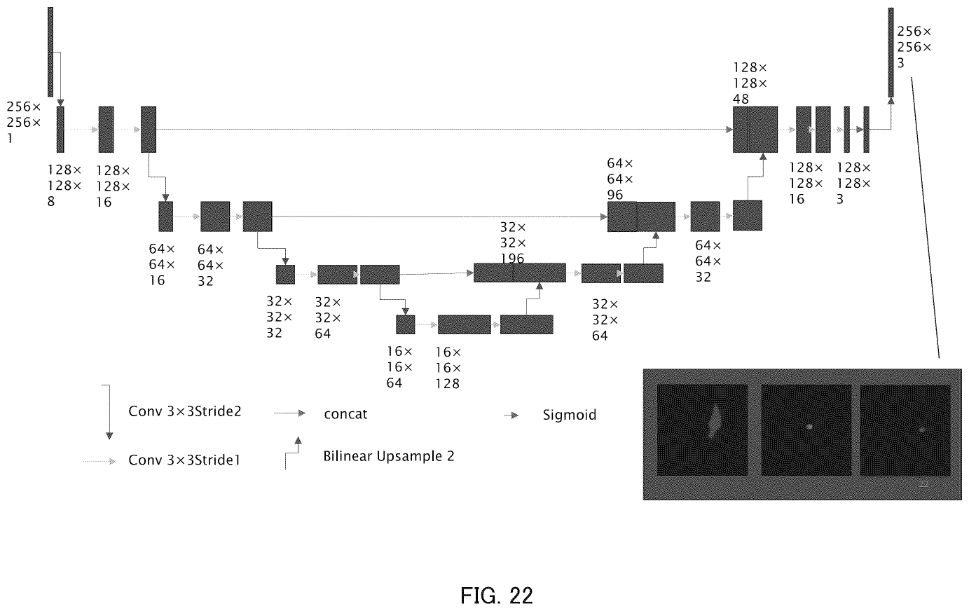

The inference section 120 inputs the ultrasound image data of an inference target to the trained machine learning model 10, and acquires, from the machine learning model 10, the left ventricular region data and the heat map data indicating the certainty factors of the positions of the left annulus end and the right annulus end. For example, when the trained machine learning model 10 is implemented as a U-net type convolutional neural network as illustrated in FIG. 22, the inference section 120 inputs ultrasound image data of an inference target to the input layer of the convolutional neural network and acquires left ventricular region data and heat map data indicating the certainty factors of the positions of the left and right annulus ends from the output layer.

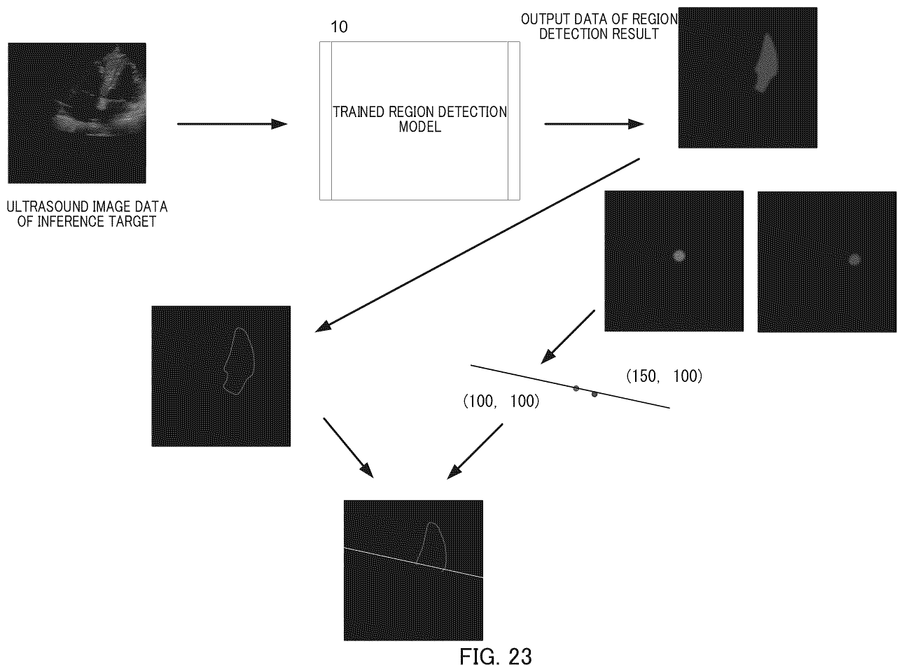

The inference section 120 determines the measurement target region based on the data indicating the left ventricular region estimated by the machine learning model 10 and the heat map data indicating the certainty factors of the positions of the left annulus end and the right annulus end. Specifically, upon acquisition of the data indicating the left ventricular region and the heat map data indicating the certainty factors of the positions of the left annulus end and the right annulus end from the machine learning model 10, as illustrated in FIG. 23, the inference section 120 derives a straight line connecting the positions with the highest certainty factors of the left annulus end and the right annulus end in the heat map data, and superimposes the straight line on the delineated left ventricular region. Then, the inference section 120 can determine a region surrounded by the rendered left ventricular region and the straight line as a measurement target region, and estimate the left ventricle ejection fraction based on the volume of the measurement target region. Further, the volume of the measurement target region may be estimated based on a contour line determined from data indicating the estimated left ventricular region.

Here, the data indicating the contour line of the left ventricular region may be acquired according to the following procedure. That is, first, the center of gravity of the left ventricular region is determined for the data indicating the left ventricular region, which is the output result from the machine learning model 10, and contour points are searched for outward from the determined center of gravity. Next, a point at which the certainty factor as an output result falls below a threshold value for the first time may be determined as a contour point. The processing variously changes an angle of the search line extending from the center of gravity, thereby determining the contour points on respective search lines. Then, these contour point data are subjected to spline interpolation to acquire a contour line. Further, the volume of the measurement target region may be estimated based on the contour line determined from the data indicating the left ventricular region in this manner. For example, the capacity of the measurement target region may be derived according to the Modified Simpson method (disk method). Specifically, the major axis (L) of two cross sections of the apical 2-chamber or 4-chamber image is equally divided into 20 disks, minor axis inner diameters (a₁ and b₁) orthogonal to the major axis are obtained, and the volume is calculated from the sum of the cross-sectional areas of the disks. Assuming that each disk has an elliptical shape, the left ventricular cavity area (V) is determined. That is, the left ventricular cavity area (V) to be measured can be calculated by the following Equation.

$$V = \frac{π}{4} \times ( {a}_{1} {b}_{1} + {a}_{2} {b}_{2} + … + {a}_{i} {b}_{i} ) \times \frac{L}{20}$$

Note that, in another example, the machine learning model 10 that directly estimates the measurement target region itself may be generated. However, estimating the measurement target region itself by the machine learning model 10 may generally degrade the estimation accuracy.

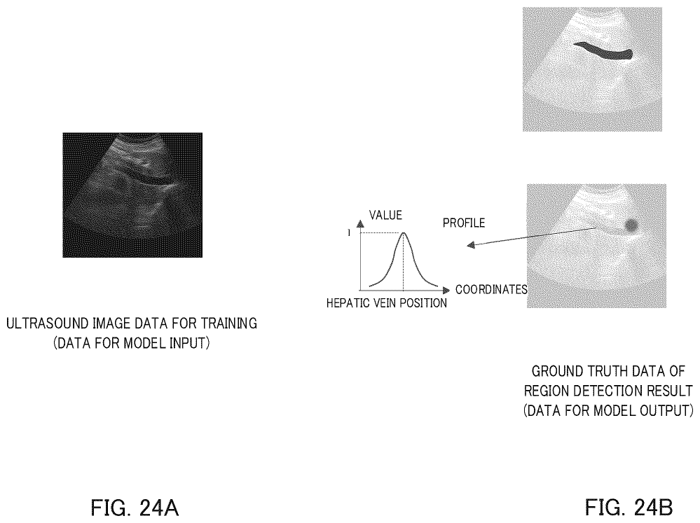

The machine learning model 10 that estimates a detection target region and a certainty factor related to a specific position described above is not limited to EF measurement, but may be used for IVC diameter measurement. First, with respect to the training processing, for example, the data acquirer 51 may acquire, from the training data DB20, ultrasound image data representing the inferior vena cava, as illustrated in FIG. 24A, as training data for input to the machine learning model 10. Furthermore, as illustrated in FIG. 24B, the data acquirer 51 may acquire, from the training data DB20, data indicating the inferior vena cava region in the ultrasound image data and heat map data indicating the certainty factor of the position of the hepatic vein, as training data to be output from the machine learning model 10. Note that the data acquirer 51 may perform preprocessing and/or data expansion on the acquired ultrasound image data for training, as necessary.

The trainer 52 may input the ultrasound image data for training to the machine learning model 10 and acquire, from the machine learning model 10, data indicating the inferior vena cava region in the ultrasound image data and heat map data indicating the certainty factor of the position of the hepatic vein. The trainer 52 compares the detection result with the ground truth data of the heat map data indicating the certainty factor of the inferior vena cava region and the position of the hepatic vein in the input ultrasound image data for training, and updates the parameters of the machine learning model 10 according to the error between the detection result and the ground truth data.

For example, in a case where the machine learning model 10 is implemented by a convolutional neural network, the trainer 52 may continue to adjust the parameters of the machine learning model 10 in accordance with the error between the output result and the ground truth data in accordance with the back propagation method until a predetermined termination condition is satisfied. After the training of the machine learning model 10 is completed in this way, the trained machine learning model 10 may be provided to the ultrasound diagnostic apparatus 100. Alternatively, the trained machine learning model 10 may be provided to a model DB40 and/or the image diagnostic apparatus 200.

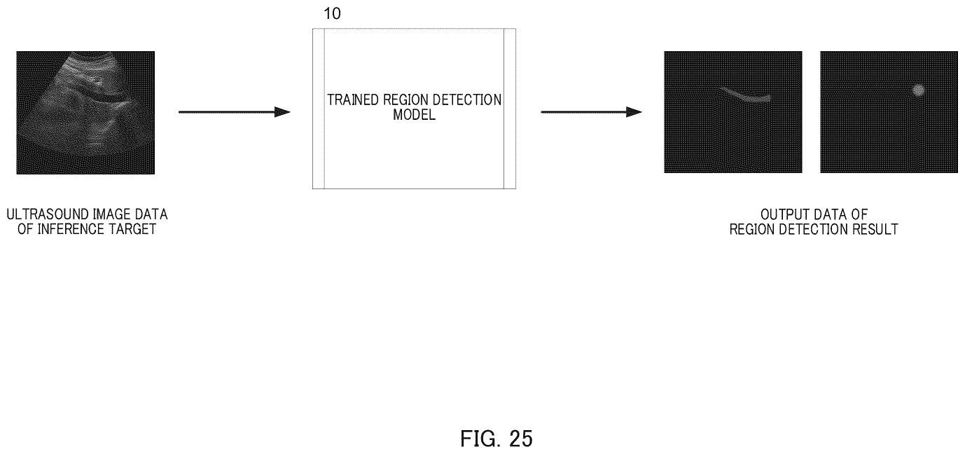

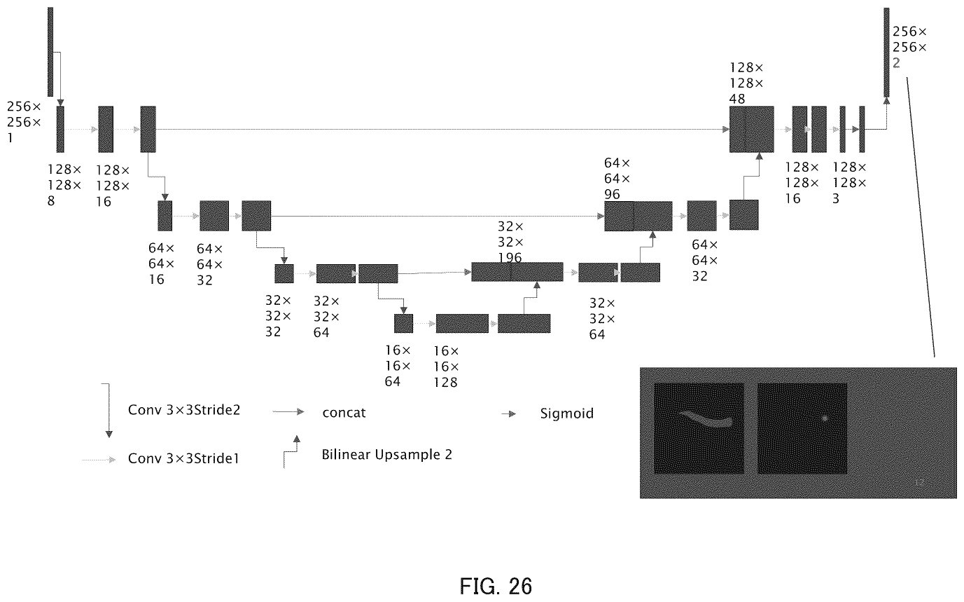

Next, in the inference processing, the data acquirer 110 acquires ultrasound image data generated based on a reception signal received from the subject 30 by the ultrasound probe 1120. As necessary, the data acquirer 110 may perform preprocessing on the acquired ultrasound image data for input to the trained machine learning mode 110. As illustrated in FIG. 25, the inference section 120 inputs the ultrasound image data of an inference target to the trained machine learning model 10 and acquires, from the machine learning model 10, an image indicating the inferior vena cava region and heat map data indicating the certainty factor of the position of the hepatic vein. For example, when the trained machine learning model 10 is implemented as a U-net type convolutional neural network as illustrated in FIG. 26, the inference section 120 inputs ultrasound image data of an inference target to the input layer of the convolutional neural network and acquires an image indicating the inferior vena cava region and heat map data indicating the certainty factor of the position of the hepatic vein from the output layer. The inference section 120 can estimate the IVC diameter from the region defined based on the image of the inferior vena cava region estimated by the machine learning model 10 and the coordinates indicating the highest certainty factor of the position of the hepatic vein.

According to the above-described example, the ultrasound diagnostic apparatus 100 may be configured to include the ultrasound probe 1120 that transmits and receives an ultrasonic wave to and from the subject 30 and an output means that outputs an inference result associated with a detection target from ultrasound image data based on a reception signal received by the ultrasound probe 1120 using the machine learning model 10.

The ultrasound diagnostic apparatus 100 may include an output means that outputs a detection region associated with a detection target as a first inference result, outputs a detection position associated with the detection target as a second inference result, and outputs a detection result associated with the detection target as a third inference result based on the detection region and the detection position from ultrasound image data based on a reception signal received by the ultrasound probe 1120 using the machine learning model 10.

Furthermore, the ultrasound diagnostic apparatus 100 may be configured to include a certainty factor generating means for generating, using the machine learning model 10, a certainty factor associated with the detection target from the ultrasound image data based on the reception signals received by the ultrasound probe 1120, and a position information acquiring means for acquiring the certainty factor maximum value coordinates based on the certainty factor. Furthermore, the ultrasound diagnostic apparatus 100 may be configured to include a shape recognition means for recognizing the shape of the detection target based on the certainty factor maximum value coordinates and an output means for outputting information on the shape of the detection target.

According to the above-described example, the ultrasound diagnostic system 1 may be configured to include a measurement position determination means configured to determine a measurement position of the detection target based on the certainty factor maximum value coordinates, a measurement means configured to measure the detection target based on the measurement position, and an output means configured to output measurement information on the measured detection target.

According to the above-described example, the machine learning model 10 may be trained using training data including ultrasound image data based on a reception signal received by an ultrasound probe, region information associated with a detection target of the ultrasound image data (e.g., a left ventricular region, an inferior vena cava, etc), and position information associated with the detection target of the ultrasound image data (e.g., coordinates of right and left annulus ends, coordinates of a hepatic vein, etc) or region information based on the position information (e.g., heat map data of the right and left annulus ends, heat map data of the hepatic vein, etc). Here, the region information based on the position information is not limited to the heat map data, but may be any type of data including the distance, from the position coordinates of the detection target, and the certainty factor. Further, the region information may be image data.

Although the examples of the present disclosure have been described in detail above, the present disclosure is not limited to the above-described specific examples, and various modifications and changes can be made within the scope of the gist of the present disclosure described in the claims.

Although embodiments of the present invention have been described and illustrated in detail, the disclosed embodiments are made for purposes of illustration and example only and not limitation. The scope of the present invention should be interpreted by terms of the appended claims.

What is claimed is:

1. A machine learning model trained by using training data that comprises:
first ultrasound image data based on a reception signal received by an ultrasound probe;
first ground truth data that is first region information associated with a detection target of the first ultrasound image data; and
second ground truth data that is first position information associated with the detection target of the first ultrasound image data or that is second region information based on the first position information.

2. The machine learning model according to claim 1, wherein the second ground truth data is the second region information.

3. The machine learning model according to claim 2, wherein the second region information is information including a distance and a certainty factor, the distance being a distance from first position coordinates associated with the detection target.

4. The machine learning model according to claim 1, wherein the first region information and the second region information are image data.

5. The machine learning model according to claim 3, wherein the second region information is first heat map information in which the information including the distance from the first position coordinates associated with the detection target and the certainty factor is converted into a heat map.

6. The machine learning model according to claim 1, wherein the training data further includes third ground truth data that is second position information associated with the detection target of the first ultrasound image data or that is third region information based on the second position information.

7. The machine learning model according to claim 1, wherein the machine learning model is composed of a convolutional neural network.

8. A non-transitory computer-readable storage medium storing a program for causing a computer to, by using the machine learning model according to claim 1, implement an output function of outputting an inference result associated with the detection target from second ultrasound image data based on the reception signal received by the ultrasound probe.

9. An ultrasound diagnostic apparatus, comprising:
an ultrasound probe that transmits and receives an ultrasonic wave to and from a subject;
an inference section that, by using the machine learning model according to claim 1, outputs an inference result associated with the detection target from second ultrasound image data based on the reception signal received by the ultrasound probe.

10. An ultrasound diagnostic apparatus comprising: an inference section that, by using a predetermined machine learning model, outputs a detection region associated with a detection target as a first inference result from second ultrasound image data based on a reception signal received by an ultrasound probe, outputs a detection position associated with the detection target as a second inference result, and outputs a detection result associated with the detection target as a third inference result based on the detection region and the detection position.

11. An ultrasound diagnostic apparatus, comprising:
an inference section that, by using a predetermined machine learning model, generates a certainty factor associated with a detection target from second ultrasound image data based on a reception signal received by an ultrasound probe and acquires certainty factor maximum value coordinates based on the certainty factor.

12. The ultrasound diagnostic apparatus according to claim 11, wherein the inference section recognizes a shape of the detection target based on the certainty factor maximum value coordinates and outputs information on the shape of the detection target.

13. The ultrasound diagnostic apparatus according to claim 11, wherein the inference section determines a measurement position of the detection target based on the certainty factor maximum value coordinates, measures the detection target based on the measurement position, and outputs measurement information on the measured detection target.

14. An ultrasound diagnostic system, comprising:
an ultrasound probe that transmits and receives an ultrasonic wave to and from a subject; and
an output that, by using the machine learning model according to claim 1, outputs an inference result associated with the detection target from second ultrasound image data based on the reception signal received by the ultrasound probe.

15. An image diagnostic apparatus, comprising: an inference section that, by using the machine learning model according to claim 1, outputs an inference result associated with the detection target from second ultrasound image data based on the reception signal received by the ultrasound probe.

16. A training apparatus that performs machine learning by using training data that comprises:
first ultrasound image data based on a reception signal received by an ultrasound probe;
first ground truth data that is first region information associated with a detection target of the first ultrasound image data; and
second ground truth data that is first position information associated with the detection target of the first ultrasound image data or that is second region information based on the first position information.
